(19) United States
(12) Patent Application Publication
(10) Pub. No.: US 2010/0179323 A1
(43) Pub. Date: Jul. 15, 2010

(54) PROCESS FOR MAKING DIPHOSPINE-RUTHENIUM-DIAMINE COMPLEXES

(76) Inventor: Paul H Moran, Missouri, TX (US)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 11/993,233

(22) PCT Filed: Jun. 28, 2006
(86) PCT No.: PCT/US06/25450

§ 371 Date: Feb. 24, 2010

Related U.S. Application Data
(60) Provisional application No. 60/696,273, filed on Jul. 1, 2005.

(51) Int. Cl. C07F 15/00; C07F 15/02; C07D 319/16

(52) U.S. Cl. 546/12; 556/23; 556/14; 549/212

(57) ABSTRACT

A process for making diphosphine-ruthenium-diamine complexes by reacting a phosphine compound with an arene ruthenium compound in a first solvent to produce an intermediate mixture comprising a diphosphine-ruthenium compound, the first solvent consisting essentially of a mixture of an aprotic solvent and a protic solvent; then removing the first solvent from the intermediate mixture to produce an intermediate solid comprising the diphosphine-ruthenium compound; and then contacting the intermediate solid comprising the diphosphine-ruthenium compound with a diamine and a second solvent to produce the diphosphine-ruthenium-diamine complex, the second solvent consisting essentially of an aprotic solvent selected from the group consisting of ethers and hydrocarbon solvents.

### BACKGROUND OF THE INVENTION

The instant invention is in the field of processes for making diphosphine-ruthenium-diamine complexes. Diphosphine-ruthenium-diamine complexes are useful, for example, as catalysts for the asymmetric hydrogenation of ketones, U.S. Pat. No. 5,763,688, and imines, U.S. Pat. No. 6,528,687. In the prior art, diphosphine-ruthenium-diamine complexes are made by reacting a diphosphine compound with an arene ruthenium chloride compound in N,N-dimethylformamide, followed by reaction of the resultant oligomeric species with a diamine to produce the diphosphine-ruthenium-diamine complex, Noyori et al., Angewandte Chemie, International Ed., 2001, 40, 40-73. However, such prior art processes for making diphosphine-ruthenium-diamine complexes require heating to above 100° C. and typically have yields of from about 50% to about 70% based on diphosphine, with the production of numerous by-products, Noyori, et al., Angewandte Chemie, International Ed., 1998, 37, 1706. Despite the significant usefulness of diphosphine-ruthenium-diamine complexes there remains a need for processes for making diphosphine-ruthenium-diamine complexes with improved yield.

### SUMMARY OF THE INVENTION

An important benefit of the process of the instant invention is the improved yield obtained thereby, through use of milder reaction conditions than the prior art process. It has been discovered that when a specific set of solvents are used diphosphine-ruthenium-diamine complexes can be produced with improved yield based on the diphosphine. More specifically, the instant invention is a process for making diphosphine-ruthenium-diamine complexes, comprising the steps of: (a) contacting a phosphine compound of formula I with an arene ruthenium compound in a first solvent to produce an intermediate mixture comprising a diphosphine-ruthenium compound of formula III, the first solvent consisting essentially of a mixture of an aprotic solvent and a protic solvent;

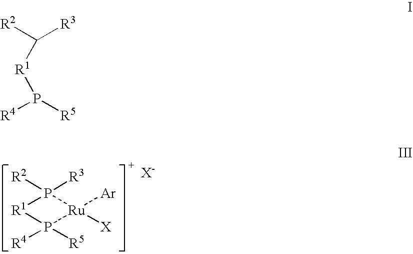

(b) removing the first solvent from the intermediate mixture to produce an intermediate solid comprising the diphosphine-ruthenium compound of formula III;
(c) contacting the intermediate solid comprising the diphosphine-ruthenium compound of formula III with a diamine of formula IV and a second solvent to produce a diphosphine-ruthenium-diamine complex of formula V, the second solvent consisting essentially of an aprotic solvent selected from the group consisting of ethers and hydrocarbon solvents,

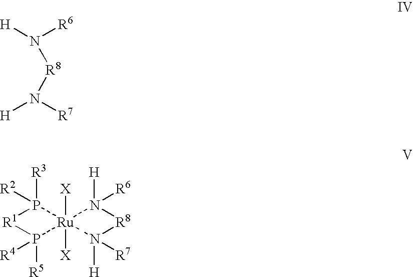

where R¹, R², R³, R⁴, R⁵, R⁶, R⁷ and R⁸ are each independently an alkyl, aryl or alkaryl group comprised of carbon, hydrogen and optionally heteroatom(s), where Ar is an aryl group comprised of carbon, hydrogen and optionally heteroatom(s) and where X is a halide or carboxylate, or any of R¹, R², R³, R⁴ and R⁵ are be linked to form cyclic chiral phosphines, or R¹ can incorporate a metallocene. Preferably, R¹, R², R³, R⁴, R⁵, R⁶, R⁷ and R⁸ comprise up to 20 carbon atoms. More preferably R¹, R², R³, R⁴, R⁵, R⁶, and R⁷ comprise up to 1, 2, 3, 4, 5, 6, 7, 8, 9, 10, 11 or 12 carbon atoms. Preferably R⁶ and/or R⁷ are hydrogen.

### DETAILED DESCRIPTION

The instant invention is a process for making diphosphine-ruthenium-diamine complexes having the formula V,

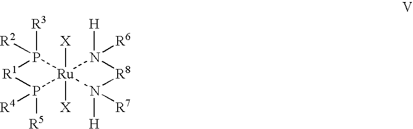

where R¹, R², R³, R⁴, R⁵, R⁶, R⁷ and R⁸ are each independently an alkyl, aryl or alkaryl group comprised of carbon, hydrogen and optionally heteroatom(s), where Ar is an aryl group comprised of carbon, hydrogen and optionally heteroatom(s) and where X is a halide or carboxylate, wherein any of R¹, R², R³, R⁴ and R⁵ can linked to form cyclic chiral phosphines, wherein R¹ can incorporate a metallocene and wherein R⁶ and/or R⁷ can be hydrogen. The compound of the following formula VI is an example of a system where R² is linked to R³ and where R⁴ is linked to R⁵. The compound of the following formula VII is an example of a system where R¹ incorporates a metallocene. The compound of the following formula VIII is an example of a system where R⁶ and R⁷ are hydrogen.

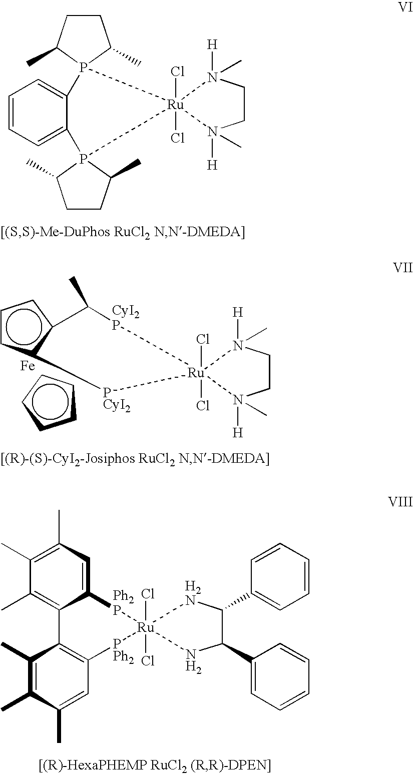

The phosphine moiety in the complex is preferably a bis-tertiary phosphine in which the two phosphorus atoms are linked by a C₂₋₇ carbon chain such that they form a 5-10 membered ring with the Ru atom. Any diamine can be used in the instant invention such as 1,2-diphenylethylene diamine (DPEN), trans-1,2-diaminocyclohexane (DACH) or even an amine substituted pyridine, for example α-picolylamine, see Ohkuma et al., J. Am. Chem. Soc., 2005, 127, 8288-8289. The diamine moiety in the complex is preferably a vicinal diamine with any aromatic, alkaryl, alkyl, heteroatom or hydrogen substituent on the carbon backbone linking the nitrogen atoms. X is preferably chloride.

The instant invention comprises three steps. The first step is to contact a phosphine compound of formula I with an arene ruthenium compound in a first solvent to produce an intermediate mixture comprising a diphosphine-ruthenium compound of formula III, the first solvent consisting essentially of a mixture of an aprotic solvent and a protic solvent. This step is preferably conducted at a temperature in the range of 0-70 degrees Celsius, more preferably in the range of 40-60 degrees Celsius and most preferably at about 55 degrees Celsius.

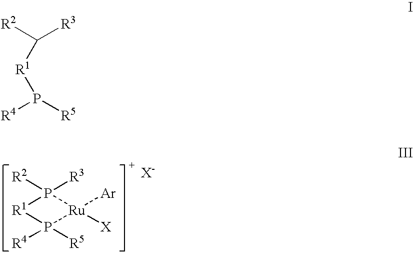

The second step is to remove the first solvent from the intermediate mixture to produce an intermediate solid comprising the diphosphine-ruthenium compound of formula III. The solvent is preferably removed by the application of a vacuum to vaporize the first solvent.

The third step is to contact the intermediate solid comprising the diphosphine-ruthenium compound of formula III with a diamine of formula IV and a second solvent to produce a diphosphine-ruthenium-diamine complex of formula V, the second solvent consisting essentially of an aprotic solvent selected from the group consisting of ethers and hydrocarbon solvents,

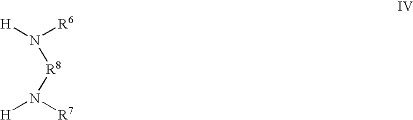

where, again, R¹, R², R³, R⁴, R⁵, R⁶, R⁷ and R⁸ are each independently in alkyl, aryl or alkaryl group comprised of carbon, hydrogen and optionally a heteroatom(s), where Ar1 is an aryl group comprised of carbon, hydrogen and optionally a heteroatom(s) and where X is a halide or carboxylate, wherein any of R¹, R², R³, R⁴ and R⁵ can linked to form cyclic chiral phosphines, wherein R¹ can incorporate a metallocene and wherein R⁶ and/or R⁷ can be hydrogen. This step is preferably conducted at a temperature in the range of 30-80 degrees Celsius, more preferably in the range of 50-70 degrees Celsius and most preferably at about 60 degrees Celsius. The compound of formula V is preferably isolated by partially removing the second solvent by vacuum assisted vaporization followed by the addition of an alcohol to crystallize the compound of formula V. The purity of the crystallized compound of formula V is preferably determined by NMR Spectroscopy.

Preferably, in the first solvent mixture the aprotic solvent consists essentially of an ether and/or a chlorinated solvent and wherein the protic solvent of the first solvent mixture consists essentially of an alcohol. More preferably, the aprotic solvent of the first solvent mixture is selected from the group consisting of diethyl ether, tetrahydrofuran, dimethyl ether, methyl-tetrahydrofuran, diisopropyl ether, methyl tert-butyl ether, di-n-butyl ether, dichloromethane and mixtures thereof and the protic solvent of the first solvent mixture is selected from the group consisting of methanol, ethanol, isopropanol, butanol and mixtures thereof. Preferably, the second solvent is selected from the group consisting of tetrahydrofuran, diethyl ether, methyl-tetrahydrofuran, diisopropyl ether, methyl tert-butyl ether, di-n-butyl ether, bis(2-methoxyethyl) ether 1,4-dioxane and mixtures thereof. The second solvent should not contain chlorinated solvents, alcohols or nitrile solvents.

In the full scope of the instant invention the arene ruthenium compound can be any monomeric or oligomeric Ru(II) complex in which each ruthenium atom is pi-bonded to a carbocyclic or heterocyclic arene. Preferably, the arene ruthenium compound is one in which the arene is a benzene, optionally forming part of a fused carbocyclic or heterocyclic ring system, and optionally bearing one or more substituents selected from the group comprising alkyl, alkenyl, alkynyl, aryl, halogen, alkoxy, acyloxy, silyloxy, aryl, amino, amido, carboxylic acid or ester, keto, or sulphonamide. A highly preferred arene is benzene or p-cymene. More preferably, the arene ruthenium compound is a dimeric complex of formula II.

[ArRuX₂]₂ II

Most preferably, the ruthenium compound is [(p-cymene)RuCl₂]₂, which has the advantage of good storage stability.

### Examples

The following examples illustrate the present invention. The complexes prepared in these examples are depicted at the end of this section. All reaction yields are based on diphosphine.

### Example 1

### Synthesis of Dichloro [(R)-4,4′,5,5′,6,6′-hexamethyl-2,2′-bis[diphenylphosphino]-biphenyl][(1R,2R)-1,2-diphenylethylenediamine]ruthenium (II): [(R)-HexaPHEMP RuCl₂(R,R)-DPEN].

A 500 ml Schlenk flask, with stirrer is charged under nitrogen with 30.2 g (49.82 mmol) of the ligand (R)-HexaPHEMP and 15.87 g of [(p-cymene)RuCl₂]₂ (25.9 mmol). 300 ml of dry and degassed methanol and 40 ml of dry and gassed dichloromethane are added. The vessel is heated to 50° C. for 30 minutes before ³¹P-NMR (CDCl₃) reveals that the reaction has gone to completion as indicated by two sets of doublets at 40.2 ppm and 27.5 ppm. The solvents are removed in vacuo to give a yellow crystalline solid. 11.63 g of (R,R)-DPEN (54.80 mmol, 1.1 eq) are added with 250 ml of dry degassed tetrahydrofuran under nitrogen. The vessel is heated to 65° C. for 8 hours before ³¹P-NMR of the crude reaction mixture reveals that the reaction has produced ˜92% product with minor by-products. The solvent is removed in vacuo to give a dry brown solid, which is treated 150 ml of dry degassed methanol at 60° C. for 30 minutes. Addition of the methanol causes, almost instantaneously, a deep yellow crystalline solid to be deposited. After cooling to room temperature the material is collected under vacuum and washed with 4×20 ml of dry degassed methanol under nitrogen. The solid is dried to yield 39.2 g (80% recovered yield) of the title compound in greater than 99% purity. ³¹P-NMR analysis of the mother liquors reveals un-recovered product and small amounts of by products.

³¹P NMR (162 MHz, CDCl₃) 545.6 ppm, singlet.

### Example 2

### Comparative synthesis of Dichloro[(R)-4,4′,5,5′,6,6′-hexamethyl-2,2′-bis[diphenylphosphino]-biphenyl][(1R,2R)-1,2-diphenylethylenediamine]ruthenium (II): [(R)-HexaPHEMP RuCl₂(R,R)-DPEN].

A 500 ml flask under nitrogen is charged with 6.07 g of (R)-HexaPHEMP (10 mmol), 100 ml dry degassed dimethylformamide and 100 ml dry degassed toluene. 3.06 g of [(p-cymene)RuCl₂]₂ (5 mmol) is added to the mixture and heated to 110° C. for 5 hours. 2.12 g of (R,R)-DPEN (10 mmol) in 100 ml of dry degassed toluene are added to the reaction and heated at 110° C. for 17 hours. The reaction mixture is cooled to room temperature (RT) before removing the dimethylformamide in vacuo. The residual dimethylformamide is removed as an azeotrope with cyclohexanone before drying to give brown oil. The brown oil is dissolved in isopropanol, after which a yellow solid is deposited and isolated by filtration and washed with 2×20 ml of isopropanol to give colourless washings. The solid is dried under vacuum to give the title compound in 65% yield.

³¹P NMR (162 MHz, CDCl₃) 545.6 ppm, singlet.

### Example 3

### Synthesis of Dichloro[(R)-2,2′-bis[di(3,5-xylyl)phosphino]-1,1′-binaphthyl][(2R)-1,1-bis(4-methoxyphenyl)-3-methyl-1,2-butanediamine]ruthenium (II): [(R)-Xyl-BINAP RuCl₂(R)-DAIPEN]

A Schlenk flask under nitrogen is charged with 50 mg of (R)-Xyl-BINAP (0.068 mmol) and 20.8 mg of [(p-cymene)RuCl₂]₂ (0.034 mmol) add charged with 5 ml of dry degassed ethanol and 0.625 ml of dry degassed dichloromethane. The solution is heated to 50° C. for 30 minutes before removing the solvent in vacuo to give a yellow crystalline solid. 21.1 mg of (R)-DAIPEN (0.068 mmol) are added with 5 ml of dry degassed tetrahydrofuran and heated at 60° C. for 8 hours. The reaction is cooled to room temperature. ³¹P NMR reveals only a single species and no starting materials. ³¹P NMR reveals >99% product and conversion. The solvent is removed in vacuo to give a pale yellow solid.

³¹P NMR (162 MHz, CDCl₃) [symbol] 46.9 ppm, doublet J_{P-P} 36.9 Hz and 44.5 ppm, doublet J_{P-P} 36.9 Hz.

### Example 4

### Synthesis of Dichloro[(S)-2,2′-bis[di(3,5-xylyl)phosphino]-1,1′-binaphthyl][(1S,2S)-1,2-diphenylethylenediamine]ruthenium (II): [(S)-Xyl-BINAP RuCl₂(S,S)-DPEN]

A Schlenk flask under nitrogen is charged with 50 mg of (S)-Xyl-BINAP (0.068 mmol) and 20.8 mg of [(p-cymene)RuCl₂]₂ (0.034 mmol) and charged with 5m1 of dry degassed ethanol and 0.625 ml of dry degassed dichloromethane. The solution is heated to 50° C. for 30 minutes before removing the solvent in vacuo to give a yellow crystalline solid. 14.4 mg of (S,S)-DPEN (0.068 mmol) are added with 5 ml of dry degassed tetrahydrofuran and heated at 60° C. for 8 hours. The reaction is cooled to room temperature. ³¹P NMR reveals only a single species and no starting materials. ³¹P NMR reveals >99% product and conversion. The solvent is removed in vacuo to give a pale yellow solid.

³¹P NMR (162 MHz, CDCl₃) [symbol] 45.3 ppm, singlet.

### Example 5

### Synthesis of Dichloro[(R)-2,2′,6,6′-Tetramethoxy-4,4′-bis(di(3,5-xylyl)phosphino)-3,3′-bipyridine][(1R,2R)-1,2-diphenylethylenediamine]ruthenium (II): [(R)-Xyl-P-Phos RuCl₂(R,R)-DPEN]

A Schlenk flask under nitrogen is charged with 100 mg of (R)-Xyl-P-Phos (0.1321 mmol) and 40.5 mg of [(p-cymene)RuCl₂ [_{’}(0.06606 mmol) and charged with 10 ml of dry degassed ethanol and 1.25 ml of dry degassed dichloromethane. The solution is heated to 50° C. for 30 minutes before removing the solvent in vacuo to give a yellow crystalline solid. 28.6 mg of (R,R)-DPEN (0.1321 mmol) are added with 10 ml of dry degassed tetrahydrofuran and heated at 60° C. for 8 hours. The reaction is cooled to room temperature. ³¹P NMR reveals only a single species and no starting materials. ³¹P NMR reveals >99% product and conversion. The solvent is removed in vacuo to give a pale yellow solid.

³¹P NMR (162 MHz, CDCl₃) [symbol] 43.8 ppm, singlet.

### Example 6

### Synthesis of Dichloro[(S)-(2,3,2′,3′-tetrahydro-5,5′-bi(1,4-benzodioxin)-6,6′-diyl)bis-(diphenylphosphane)][(1S,2S)-1,2-diphenylethylenediamine]ruthenium (II): [(S)-SYNPHOS RuCl₂(S,S)-DPEN]

A Schlenk flask under nitrogen is charged with 50 mg of (S)-SYNPHOS (0.0783 mmol) and 23.9mg of [(p-cymene)RuCl₂]₂ (0.0392 mmol) and charged with 5 ml of dry degassed ethanol and 0.625 ml of dry degassed dichloromethane. The solution is heated to 50° C. for 30 minutes before removing the solvent in vacuo to give a yellow crystalline solid. 16.6 mg of (S,S)-DPEN (0.068 mmol) are added with 5 ml of dry degassed tetrahydrofuran and heated at 60° C. for 8 hours. The reaction is cooled to room temperature. ³¹P NMR reveals only a single species and no starting materials. ³¹P NMR reveals >99% product and conversion. The solvent is removed in vacuo to give a pale yellow amorphous solid.

³¹P NMR (162 MHz, CDCl₃) [symbol] 47.2 ppm, singlet.

### Example 7

### Synthesis of Dichloro[(R)-(6,6′-O-(1,4-butylene)-oxylbiphenyl-2.2′-diyl)bis(diphenyl)phosphine][(1R,2R)-1,2-diphenylethylenediamine]ruthenium (II): [(R)-C₄-TunePHOS RuCl₂(R,R)-DPEN]

A Schlenk flask under nitrogen charged with 50 mg of (R)-C₄-TunePHOS (0.0822 mmol) and 25.1 mg of [(p-cymene)RuCl₂]₂ (0.0411 mmol) and charged with 5 ml of dry degassed ethanol and 0.625 ml of dry degassed dichloromethane. The solution is heated to 50° C. for 30 minutes before removing the solvent in vacuo to give a yellow crystalline solid. 17.4 mg of (R,R)-DPEN (0.068 mmol) are added with 5 ml dry degassed tetrahydrofuran and heated at 60° C. for 8 hours. The reaction is cooled to room temperature. ³¹P NMR reveals ˜80% product and 20% of a by-product. The solvent is removed in vacuo and the residue treated with isopropanol to give a pale yellow amorphous solid which is isolated by filtration to give the title compound.

³¹P NMR (162 MHz, CDCl₃) [symbol] 47.95 ppm, singlet.

### Example 8

### Synthesis of [(S)-2,2′-bis[diphenylphosphino]-1,1′-binaphthyl][(1R,2R)-1,2-diaminocyclohexane]ruthenium (II): [(S)-BINAP RuCl₂(R,R)-DACH]

A Schlenk flask under nitrogen is charged with 101.7 mg of (S)-BINAP (0.1633 mmol) and 50 mg of [(p-cymene)RuCl₂]₂ (0.0816 mmol) and charged with 5 ml of dry degassed dichloromethane and 0.625 ml of dry degassed methanol. The solution is heated to 50° C. for 20 minutes before removing the solvent in vacuo to give a yellow crystalline solid. 18.65 mg of (R,R)-DACH (0.1633 mmol) are added with 5 ml of dry degassed tetrahydrofuran and heated at 60° C. for 4 hours. The reaction is cooled to room temperature. ³¹P NMR reveals essentially a single species and no starting materials. ³¹P NMR reveals >95% product and conversion. The solvent is removed in vacuo to give a pale yellow amorphous solid.

³¹P NMR (162 MHz, CDCl₃) [symbol] 46.42 ppm, singlet.

### Example 9

### Synthesis of [(S)-2,2′-bis[di(4-methylphenyl)phosphino]-1,1′-binaphthyl][(1R,2R)-1,2-diaminocyclohexane]ruthenium (II): [(S)-Tol-BINAP RuCl₂(R,R)-DACH]

A Schlenk flask under nitrogen is charged with 110.8 mg of (S)-BINAP (0.1633 mmol) and 40.8 mg of [(benzene)RuCl₂]₂ (0.0816 mmol) and charged with 5 ml of dry degassed dichloromethane and 0.625 ml of dry degassed methanol. The solution is heated to 50° C. for 15 minutes before removing the solvent in vacuo to give a yellow crystalline solid. 18.65 mg of (R,R)-DACH (0.1633 mmol) are added with 5 ml of dry degassed tetrahydrofuran and heated at 60° C. for 4 hours. The reaction is cooled to room temperature. ³¹P NMR reveals essentially a single species and no starting materials. ³¹P NMR reveals >96% product and conversion. The solvent is removed in vacuo to give a pale yellow amorphous solid.

³¹P NMR (162 MHz, CDCl₃) [symbol] 44.79 ppm, singlet.

### Example 10

### General Procedure and Application to the Preparation to Additional Diphosphine-Ruthenium-Diamine Complexes

A Schlenk flask under nitrogen is charged with 0.1631 mmol of phosphine ligand and 50.0 mg of [(p-cymene)RuCl₂]₂ (0.0816 mmol) and charged with a first solvent consisting of 5 ml of dry degassed ethanol and 0.625 ml of dry dichloromethane. The solution is heated to 50° C. for 15 minutes before removing the solvent in vacuo to give yellow crystalline solids. 14.4 mg of N,N′-DMEDA (0.1631 mmol) are added with 5 ml of dry degassed tetrahydrofuran (second solvent) and heated at 60° C. The reaction is cooled to room temperature and analysed by ³¹P NMR The solvents are removed in vacuo and the residue treated with isopropanol to give a pale yellow—yellow/brown solids, which are isolated by filtration. This method is used to prepare the following complexes:
Dichloro[rac-bis[di(3,5-xylyl)phosphino]-1,1′-binaphthyl][1,2-N,N′-dimethyl-ethylenediamine]ruthenium (II):
[rac-Xyl-BINAP RuCl₂N,N′-DMEDA]
Dichloro[rac-6,6′-difluoro-2,2′-bis[diphenylphosphino]-biphenyl][1,2-N,N′-dimethyl-ethylenediamine]ruthenium (II): [rac-F-BIPHEP RuCl₂N,N′-DMEDA]
Dichloro[1,2-Bis-((2S,5S)-2,5-dimethylphospholano)benzene][1,2-N,N′-dimethyl-ethylenediamine]ruthenium (II): [(S,S)-Me-DuPhos RuCl₂N,N′-DMEDA]
Dichloro[(R)-(−)-1-[(S)-2-(dicyclohexylphosphino)ferrocenyl]ethyldicyclohexylphosphine][1,2-N,N′-dimethyl-ethylenediamine]ruthenium (II): [(R)-(S)-(Cyl)₂-Josiphos RuCl₂N,N′-DMEDA]

The following table shows reaction times, conversion and NMR characterisation data for each of the complexes:

| | | Reaction | Stage 1 | Reaction | | Stage 2 |
|---|---|---|---|---|---|---|
| | | time | ³¹P-NMR | time | ³¹P-NMR | ³¹P-NMR |
| Phosphine | Diamine | Stage 1 | Data | Stage 2 | Conversion | Data CDCl₃ |
| rac-Xyl- | N,N′- | 20 minutes | 39.5 ppm, | 120 | >99% | 41.54 ppm |
| BINAP | DMEDA | | doublet, | minutes | | singlet |
| | | | 62.2 Hz; | | | |
| | | | 27.8 ppm | | | |
| | | | doublet, | | | |
| | | | 62.2 Hz | | | |
| rac-F- | N,N′- | 20 minutes | 41.3 ppm, | 5 hours | >99% | 40.55 ppm |
| BIPHEP | DMEDA | | doublet, | | | singlet |
| | | | 65.3 Hz; | | | |
| | | | 27.9 ppm | | | |
| | | | doublet, | | | |
| | | | 65.3 Hz | | | |
| (S,S)- | N,N′- | 20 minutes | 97.4 ppm, | 48 hours | >90% | 88.9 ppm |
| MeDuPhos | DMEDA | | doublet, | | | singlet |
| | | | 36.1 Hz; | | | |
| | | | 83.9 ppm | | | |
| | | | doublet, | | | |
| | | | 36.1 Hz | | | |
| (R)-(S)- | N,N′- | 20 minutes | 55.9 ppm, | 5 hours | >95% | 58.6 ppm, |
| (Cyl)₂- | DMEDA | | doublet, | | | doublet, |
| Josiphos | | | 30.2 ppm | | | 37.6 Hz; |
| | | | doublet, | | | 41.6 ppm, |
| | | | | | | doublet, |
| | | | | | | 37.6 Hz. |

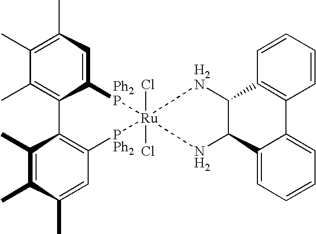

[(R)-HexaPHEMP RuCl₂ (R,R)-DPEN]

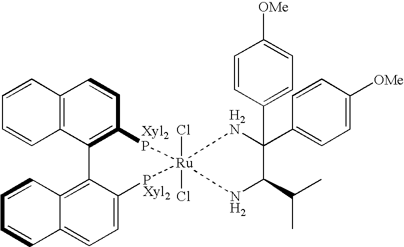

[(R)-Xyl-BINAP RuCl₂ (R)-DAIPEN]

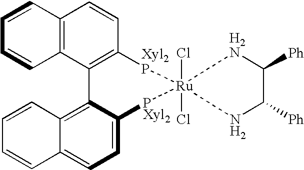

[(S)-Xyl-BINAP RuCl₂ (S,S)-DPEN]

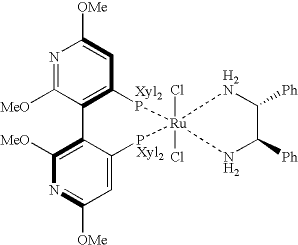

[(R)-Xyl-P-PHOS RuCl₂ (R,R)-DPEN]

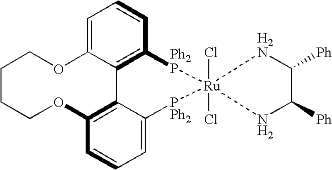

[(R)-C₄-TunePHOS RuCl₂ (R,R)-DPEN]

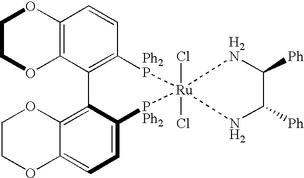

[(S)-SynPHOS RuCl₂ (S,S)-DPEN]

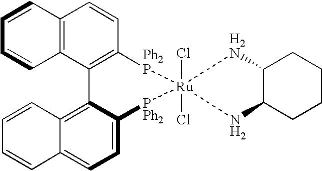

[(S)-BINAP RuCl₂ (R,R)-DACH]

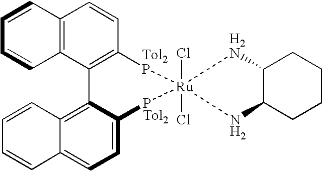

[(S)-Tol-BINAP RuCl₂ (R,R)-DACH]

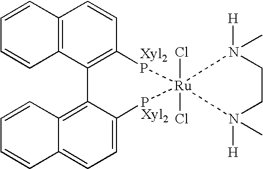

[(rac)-Xyl-BINAP RuCl₂ N,N′-DMEDA]

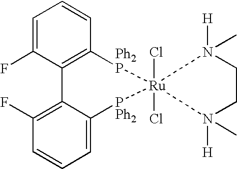

[(rac)-F-BIPHEP RuCl₂ N,N′-DMEDA]

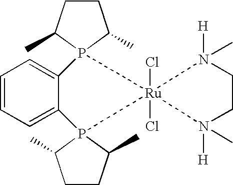

[(S,S)-Me-DuPhos RuCl₂ N,N′-DMEDA]

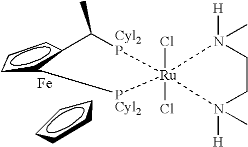

[(R)-(S)-Cyl₂-Josiphos RuCl₂ N,N′-DMEDA]

What is claimed is:

1. A process for making diphosphine-ruthenium-diamine complexes, comprising the steps of: (a) contacting a phosphine compound of formula I with an arene ruthenium compound in a first solvent to produce an intermediate mixture comprising a diphosphine-ruthenium compound of formula III, the first solvent consisting essentially of a mixture of an aprotic solvent and a protic solvent; 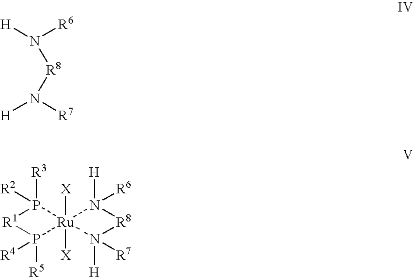
(b) removing the first solvent from the intermediate mixture to produce an intermediate solid comprising the diphosphine-ruthenium compound of formula III;
(c) contacting the intermediate solid comprising the diphosphine-ruthenium compound of formula III with a diamine of formula IV and a second solvent to produce a diphosphine-ruthenium-diamine complex of formula V, the second solvent consisting essentially of an aprotic solvent selected from the group consisting of ethers and hydrocarbon solvents, 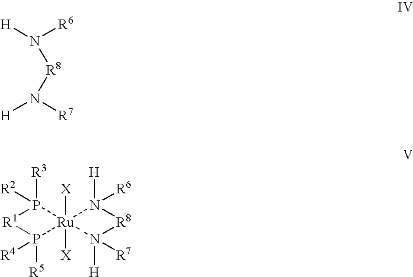
where R¹, R², R³, R⁴, R⁵, R⁶, R⁷ and R⁸ are each independently an alkyl, aryl or alkaryl group comprised of carbon, hydrogen and optionally heteroatom(s), where Ar is an aryl group comprised of carbon, hydrogen and optionally heteroatom(s) and where X is a halide or carboxylate or wherein any of R¹, R², R³, R⁴ and R⁵ are linked to form cyclic chiral phosphines, or R¹ can incorporate a metallocene.

2. The process of claim 1, wherein the compound of formula I is a bis-tertiary phosphine in which the two phosphorus atoms are linked by a C₂₋₇ carbon chain such that they form a 5-10 membered ring with the Ru atom of the compound of formula III, wherein the compound of formula IV is a chelating diamine with any aromatic, alkaryl, alkyl, heteroatom or hydrogen substituent on the carbon backbone linking the nitrogen atoms and wherein X is chloride.

3. The process of claim 1, wherein the first solvent mixture consists essentially of an ether and/or a chlorinated solvent and wherein the protic solvent of the first solvent mixture consists essentially of an alcohol.

4. The process of claim 2, wherein the first solvent mixture consists essentially of an ether and/or a chlorinated solvent and wherein the protic solvent of the first solvent mixture consists essentially of an alcohol.

5. The process of claim 1, wherein the aprotic solvent of the first solvent mixture is selected from the group consisting of diethyl ether, tetrahydrofuran, dimethyl ether, methyl-tetrahydrofuran, diisopropyl ether, methyl tert-butyl ether, di-n-butyl ether, dichloromethane and mixtures thereof and the protic solvent of the first solvent mixture is selected from the group consisting of methanol, ethanol, isopropanol, butanol and mixtures thereof.

6. The process of claim 2, wherein the aprotic solvent of the first solvent mixture is selected from the group consisting of diethyl ether, tetrahydrofuran, dimethyl ether, methyl-tetrahydrofuran, diisopropyl ether, methyl tert-butyl ether, di-n-butyl ether, dichloromethane and mixtures thereof and the protic solvent of the first solvent mixture is selected from the group consisting of methanol, ethanol, isopropanol, butanol and mixtures thereof.

7. The process of claim 1, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

8. The process of claim 2, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

9. The process of claim 3, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

10. The process of claim 4, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

11. The process of claim 5, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

12. The process of claim 6, wherein the second solvent is selected from the group consisting of tetrahydrofuran, diethyl ether, methyl-tetrahydrofuran, diisopropyl ether, methyl tert-butyl ether, di-n-butyl ether and mixtures thereof.

13. The process of any of claims 1-12, wherein the diamine is a a vicinal diamine with any aromatic, alkaryl, alkyl, heteroatom or hydrogen substituent on the carbon backbone linking the nitrogen atoms.

14. The process of claim 13, wherein the vicinal diamine is 1,2-diphenylethylene diamine (DPEN) or trans-1,2-diaminocyclohexane (DACH).

15. The process of any of claims 1-12, wherein the diamine is an amine substituted pyridine.

16. The process of any of claims 1-15, wherein the arene ruthenium compound is a monomeric or oligomeric Ru(II) complex in which each ruthenium atom is pi-bonded to a carbocyclic or heterocyclic arene.

17. The process of claim 16, wherein the arene ruthenium compound is one in which the arene is a benzene, optionally forming part of a fused carbocyclic or heterocyclic ring system, and optionally bearing one or more substituents selected from the group comprising alkyl, alkenyl, alkynyl, aryl, halogen, alkoxy, acyloxy, silyloxy, aryl, amino, amido, carboxylic acid or ester, keto, or sulphonamide.

18. The process of claim 16, wherein the arene of the arene ruthenium compound is benzene or p-cymene.

19. The process of claim 16, wherein the arene ruthenium compound is a dimeric complex of formula II.
[ArRuX₂]₂ II

20. The process of claim 19, wherein the ruthenium compound is [(p-cymene)RuCl₂]₂.
